(19) United States
(12) Patent Application Publication
(10) Pub. No.: US 2025/0025312 A1
(43) Pub. Date: Jan. 23, 2025

(54) ORTHOPEDIC KNEE JOINT PROSTHESIS

(71) Applicant: Aesculap AG, Tuttlingen (DE)

(72) Inventors: Arthur Bollinger, Tuttlingen (DE); Stephanie Stoll, Villingendorf (DE); Jana Gerlitz, Roth (DE)

(21) Appl. No.: 18/762,992

(22) Filed: Jul. 3, 2024

(30) Foreign Application Priority Data
Jul. 17, 2023 (DE) 10 2023 118 837.2

(51) Int. Cl. A61F 2/38; A61F 2/30

(52) U.S. Cl. CPC A61F 2/3859; A61F 2/30965; A61F 2/389

(57) ABSTRACT

An implant part for an orthopedic knee joint prosthesis has a surface region which faces and contacts bone in an implanted state. A least part of the implant part is made from a plastics material, in particular polyether ether ketone or polyether ether ketone composite material. The implant part has a three-dimensional surface structure in the surface region facing the bone. The three-dimensional surface structure includes a rib structure, a first rib portion extending in a plane on a surface of the surface region, or a first rib of the rib structure extending in the plane on the surface of the surface region, and a second rib portion extending in the plane on the surface of the surface region, or a second rib of the rib structure extending in the plane on the surface of the surface region extending in the plane in different directions, at least in portions.

### CROSS-REFERENCE TO RELATED APPLICATION

This application claims priority under 35 U.S.C. § 119 to German Application No. 10 2023 118 837.2, filed on Jul. 17, 2023, the content of which is incorporated by reference herein in its entirety.

### FIELD

The present disclosure relates to a femoral implant part for an orthopedic knee joint prosthesis, the femoral implant part having at least one surface region which faces a bone and is in contact with the bone in the implanted state. The present disclosure also relates to a tibial implant part for an orthopedic knee joint prosthesis, and to an orthopedic knee joint prosthesis, and to a production method for producing the implant parts or the orthopedic knee joint prosthesis.

### BACKGROUND

Femoral and tibial implant parts and such knee joint prostheses are known from the prior art. Materials used for such implants consist substantially of cobalt-chromium or cobalt-chromium-molybdenum (Co—Cr—Mo) alloys or ceramic materials. The prosthesis is generally fastened directly to the bone using bone cement or by bone engraftment.

Metal or ceramic materials cause some unwanted clinical problems. Due to comparatively good strength and stability and good biological compatibility of polyether ether ketone (PEEK) polymer materials, it is also known, for example, to produce implants from polyether ether ketone (PEEK) polymer materials.

Due to the comparatively high impact load, the very complex surface shape of the knee joint and the relatively low degree of adaptation, the pressure on the surface of the knee joint (10 to 20 MPa) is comparatively high, for example much higher than in a hip joint (2 to 5 MPa).

These relatively high mechanical environmental conditions and requirements lead to considerable wear of such an implant.

### SUMMARY

The object of the present disclosure is to provide a femoral implant part for an orthopedic knee joint prosthesis, or an orthopedic knee joint prosthesis, by means of which the aforementioned disadvantages can be overcome.

One embodiment relates to a femoral implant part for an orthopedic knee joint prosthesis, the femoral implant part having at least one surface region which faces the bone and is in contact with the bone in the implanted state, at least part of the femoral implant part, at least the surface region in contact with the bone, being made from a plastics material, in particular polyaryl ether ketone, (PAEK), in particular polyether ether ketone (PEEK) or polyether ether ketone (PEEK) composite material, and the femoral implant part comprising a three-dimensional surface structure in the surface region facing the bone, and the three-dimensional surface structure comprises at least one rib structure, at least one first rib portion extending on a surface of the surface region in one plane or a first rib of the rib structure extending on the surface of the surface region in the plane, and a second rib portion extending on the surface of the surface region in the plane, or a second rib of the rib structure extending on the surface of the surface region in the plane extending in different directions, at least in portions.

The rib structure is thus formed on a surface of the surface region, and thus forms the three-dimensional surface structure. The rib structure comprises at least two or more ribs and/or at least two or more rib portions. These ribs or rib portions are arranged or formed on the surface of the surface region. The ribs or rib portions thus project from the surface of the surface region, proceeding therefrom, or protrude from the surface. A rib structure is understood to mean a strip-shaped structure which is curved starting from the surface of the surface region.

The ribs or rib portions project macroscopically by at least 0.5 mm and in particular up to 3 mm from a surface of the surface region of the implant part. In the plane in which the ribs or rib portions extend, these have a width of at least 0.5 mm and in particular up to 3 mm. In the plane, the ribs or rib portions are of any desired length, in particular at least 1 mm. A maximum length is limited, for example, by a specific arrangement of a plurality of ribs or rib portions, and/or by an extension of the surface region.

Two rib portions can be associated with a common rib or two different ribs. A rib and a rib portion can in principle extend in any manner in the plane, in particular straight, but also at an angle or curved.

The rib structure provided on the surface increases the stability of the femoral implant. The improvement in stability is advantageous because an implant made of plastics material, in particular polyether ether ketone (PEEK) or polyether ether ketone (PEEK) composite material, has lower strength values than a comparable implant made of metal, for example of a cobalt-chromium alloy.

In principle, the femoral implant part can be anchored to the bone without cement or using bone cement.

In addition, the provided rib structure can be advantageous for improving a cemented anchoring of the implant with the bone.

It is provided, for example, that the three-dimensional surface structure comprises at least one, advantageously a plurality of, intermediate space(s) formed between at least two ribs and/or rib portions of the rib structure. An intermediate space is delimited on one side by the surface of the surface region and by the ribs or rib portions toward a corresponding side. An intermediate space is open on a side facing the bone in the implanted state, and thus forms a space for receiving the cement for the cemented anchoring of the implant.

According to one embodiment, it is provided that, in relation to a reference axis extending in the plane, a rib or a rib portion extends in the plane longitudinally with respect to the reference axis, or transversely to the reference axis, or obliquely, in particular at an angle of greater than 0° and less than 180° with respect to the reference axis, or curved or angled.

According to one embodiment, it is provided that at least two or more ribs or at least two or more rib portions of the rib structure form at least one intersection, in particular in the form of a T-intersection, Y-intersection, +-intersection or star intersection. Such intersections can increase the stability or strength of the rib structure, and thus improve the strength of the femoral implant.

According to one embodiment, it is provided that ribs or rib portions of the rib structure are arranged according to a honeycomb pattern such that in each case six ribs or rib portions delimit one cell of the honeycomb pattern. In the case of a uniform honeycomb shape, the ribs or rib portions of a cell meet at an angle of 120°. Deviations from an ideal honeycomb shape are also conceivable. Alternatively, a pattern similar to a honeycomb pattern can also be formed, for example the cells can also be delimited by a triangle or pentagon, as well as by three or five ribs or rib portions in each case, or seven or more ribs or rib portions. A rib structure which is designed according to such a pattern, in particular a honeycomb pattern, has a particularly advantageous degree of strength or stability.

According to one embodiment, it is provided that a rib or a rib portion has a spiral profile in the plane. In this connection, it can be advantageous if the surface region is completely surrounded in an edge region by one or more ribs or rib portions. In this way, a space for receiving cement is provided.

The object mentioned at the outset is also achieved by a tibial implant part for an orthopedic knee joint prosthesis. The tibial implant part has at least one surface region that faces the bone and is in contact with the bone in the implanted state. At least part of the tibial implant part, at least the surface region in contact with the bone, is made from a plastics material, in particular polyether ether ketone (PEEK) or polyether ether ketone (PEEK) composite material. The tibial implant part has a three-dimensional surface structure in the surface region facing the bone, and the three-dimensional surface structure comprises at least one rib structure, at least one first rib portion extending in a plane on a surface of the surface region, or a first rib of the rib structure extending in the plane on the surface of the surface region, and a second rib portion extending in the plane on the surface of the surface region, or a second rib of the rib structure extending in the plane on the surface of the surface region, extending in the plane in different directions, at least in portions.

According to further embodiments, the tibial implant part, or the surface region of the tibial implant part, is designed analogously to the surface region described in relation to the femoral implant part.

It is provided, for example, that the three-dimensional surface structure comprises at least one, advantageously a plurality of, intermediate space(s) formed between at least two ribs and/or rib portions of the rib structure.

According to one embodiment, it is provided that, in relation to a reference axis extending in the plane, a rib or a rib portion extends in the plane longitudinally with respect to the reference axis, or transversely to the reference axis, or obliquely, in particular at an angle of greater than 0° and less than 180° with respect to the reference axis, or curved or angled.

According to one embodiment, it is provided that at least two or more ribs or at least two or more rib portions of the rib structure form at least one intersection, in particular a T-intersection, Y-intersection, +-intersection or star intersection.

According to one embodiment, it is provided that ribs or rib portions of the rib structure are arranged according to a honeycomb pattern such that in each case six ribs or rib portions delimit a cell of the honeycomb pattern.

According to one embodiment, it is provided that a rib or a rib portion has a spiral profile in the plane. In this connection, it can be advantageous if the surface region is completely surrounded in an edge region by one or more ribs or rib portions. The object mentioned at the outset is also achieved by an orthopedic knee joint prostheses. Such an orthopedic knee joint prosthesis comprises a femoral implant part according to the described embodiments and/or a tibial implant part according to the described embodiments, and optionally a meniscus replacement part which forms a sliding partner for the femoral implant part and is held on the tibial implant part.

Further embodiments relate to a method for producing a femoral implant part according to the described embodiments and/or a tibial implant part according to the described embodiments. It is provided, for example, that the femoral implant part and/or the tibial implant part is/are produced in an injection molding process or in an additive manufacturing process. In an injection molding process, the three-dimensional surface structure is produced by injecting material into a corresponding mold cavity. In an additive manufacturing method—3D printing—the three-dimensional surface structure is produced by the layered addition of material on the surface.

Further features, details and advantages of the present disclosure can be found in the accompanying drawings and in the following description of an embodiment according to the present disclosure of the orthopedic knee joint prosthesis. In this case, the same reference signs in different drawings denote the same or at least functionally comparable elements. In the description of individual drawings, reference may also be made to elements from other drawings.

### BRIEF DESCRIPTION OF THE DRAWINGS

In the drawings:

FIG. 1 is an exploded view of an embodiment according to the present disclosure of an orthopedic knee joint prosthesis comprising a femoral implant part, a tibial implant part, and a meniscus replacement part fixable to the tibial implant part;

FIG. 2 shows a femoral implant part for an orthopedic knee joint prosthesis according to a first embodiment;

FIG. 3 shows a femoral implant part for an orthopedic knee joint prosthesis according to a further embodiment;

FIG. 4 shows a femoral implant part for an orthopedic knee joint prosthesis according to a further embodiment;

FIG. 5 shows a three-dimensional surface structure according to a first embodiment;

FIG. 6 shows a three-dimensional surface structure according to a further embodiment;

FIG. 7 shows a three-dimensional surface structure according to a further embodiment; and

FIG. 8 is a plan view from below of a tibial implant part.

### DETAILED DESCRIPTION

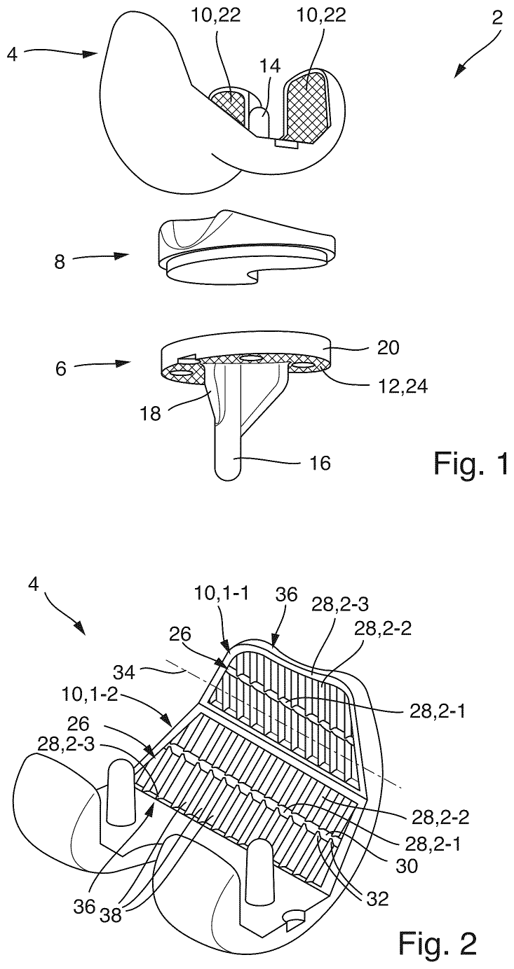

FIG. 1 shows, in an exploded view, an orthopedic knee joint prosthesis, denoted as a whole by reference sign 2, comprising a femoral implant part 4, a tibial implant part 6, and a meniscus replacement part 8, which can typically be anchored to the tibial implant part 6. The femoral implant part 4 comprises a surface region 10 that faces a bone (not shown) of the thigh (femur) and is in contact with the bone.

The tibial implant part 6 also has a surface region 12 that faces the bone of the lower leg (tibia) and is in contact with the bone.

The femoral implant part 4 has at least one dome-shaped or shaft-shaped projection 14, and the tibial implant part 6 has an elongate projection 16, which each serve for engagement and anchoring on the thigh or lower leg bone, respectively. The projection 16 of the tibial implant part 6 is also attached to a disk-shaped plateau part 20 of the tibial implant part 6, via cheek-like or wing-shaped portions 18 extending in the vertical plane.

In the preferred case shown by way of example, the femoral implant part 4 and the tibial implant part 6 are each designed having a three-dimensional surface structure 22 and 24, respectively, in the region of their bone contact, namely the surface region 10, 12. In both cases, this three-dimensional surface structure 22, 24 projects macroscopically by at least 0.5 mm and in particular up to 3 mm from a surface of the surface region 10, 12 of the implant part 4 or 6.

With reference to FIG. 2 to 7, various advantageous embodiments of the three-dimensional surface structure 22 are explained below, using the example of the femoral implant part 4. The three-dimensional surface structure 24 of the tibial implant part 6 can accordingly be designed analogously.

FIG. 2 shows a femoral implant part 4. In the example, the femoral implant part 4 comprises a plurality of surface regions 10 in contact with the bone, in which a three-dimensional surface structure 14 is formed. In the example, the surface regions 10-1, and 10-2 are shown. Further surface regions, for example in the region of the femoral condyles, are not visible due to the view shown. Three-dimensional surface structures (not shown) can also be formed on the other surface regions.

A surface structure 14 of the surface regions 10-1 and 10-2 comprises a rib structure 26. A rib structure of this kind comprises ribs 28 or rib portions 30. A rib 28 is understood to mean, for example, a strip-shaped structure which extends without interruption and bulges starting from the surface. A rib portion 30 is, for example, any portion of such a rib 28. In particular, a rib portion 30 is a portion extending from an intersection 32 at which at least two ribs 18 intersect or meet, as far as another intersection 32.

In the embodiment according to FIG. 2, the rib structure 26 of the surface regions 10-1 and 10-2 comprises ribs 28-1 extending longitudinally with respect to a reference axis 34, and ribs 28-2 extending transversely to the reference axis 34. Both surface regions 10-1 and 10-2 are completely surrounded in an edge region 36 by a circumferential rib 28-3.

Alternatively, ribs 28 or rib portions 30 can also extend obliquely to the reference axis 34.

In the example shown according to FIG. 2, longitudinally and transversely extending ribs 28-1, 28-2 meet one another at intersections 22 at a right angle. The intersections shown are therefore in the form of a +-intersection. Alternatively, ribs 28 can also cross or meet at any other angles or a different number of ribs can cross or meet Further possible intersection forms are, for example, T-intersection, Y-intersection, star intersection, or any other intersection forms.

In the example shown according to FIG. 2, a plurality of intermediate spaces 38 are formed between at least ribs 28, 28-1, 28-2, 28-3 or rib portions 30 of the rib structure 26. An intermediate space 38 is delimited on one side by the surface of the surface region 10, 10-1, 10-2 and by the ribs 28, 28-1, 28-2, 28-3 or rib portions 30 toward a corresponding side. An intermediate space 38 is open on a side facing the bone in the implanted state.

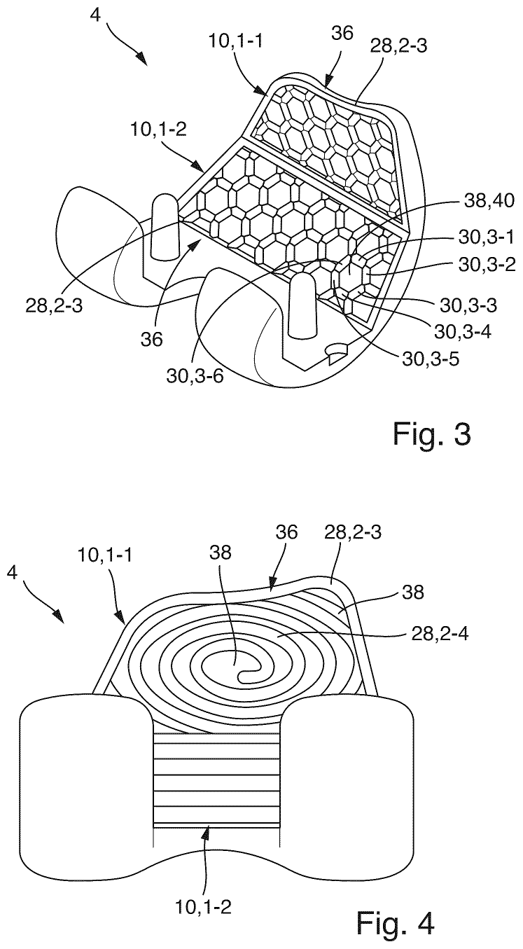

FIG. 3 shows a further embodiment. In the example, the surface regions 10-1, and 10-2 are shown. A surface structure 14 of the surface regions 10-1 and 10-2 comprises a rib structure 26. The surface structure 14 comprises ribs 28 or rib portions 30, which are arranged according to a honeycomb pattern. In the example, six rib portions 30-1, 30-2, 30-3, 30-4, 30-5, 30-6 delimit a cell 40 of the honeycomb pattern.

A cell 40 of the honeycomb pattern forms an intermediate space 38.

FIG. 4 shows a further embodiment. In the example, the surface regions 10-1, and 10-2 are shown. A surface structure 14 of the surface regions 10-1 and 10-2 comprises a rib structure 26.

The rib structure 26 of the surface region 10-1 comprises a rib 28-4 having a spiral profile. The rib 28-4 comprises a plurality of rib portions 30 which, due to the spiral profile in the plane, extend at least in portions in different directions. The surface region 10-1 is completely surrounded in an edge region 36 by a circumferential rib 28-3.

The rib structure 26 of the surface region 10-1 comprises a plurality of ribs 28-1 extending longitudinally with respect to a reference axis 34.

The surface region 10-2 is also completely surrounded, in an edge region 36, by a circumferential rib 28-3.

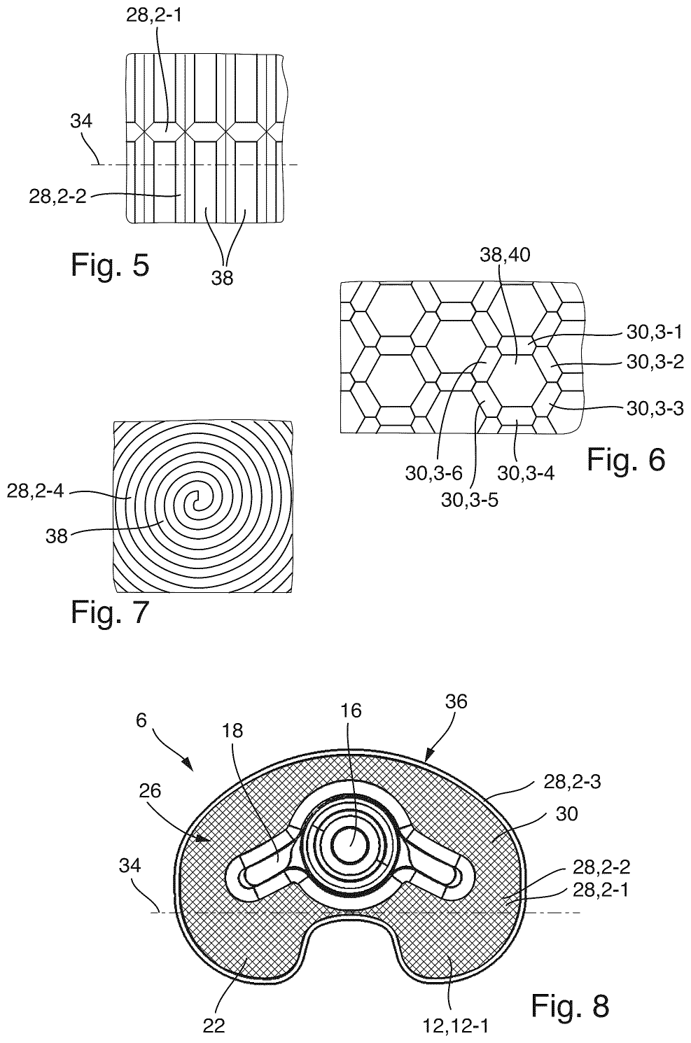

FIG. 5, 6, 7 show cutouts of the three-dimensional surface structures from FIG. 2, 3, 4.

In principle, in the case of a femoral implant 4, a combination of the illustrated three-dimensional surface structures or other three-dimensional surface structures in different surface regions is possible. A combination within a particular surface region is also possible. In a surface region, a surface structure or an arrangement of the ribs or rib portions or a pattern does not necessarily have to extend over an entire surface region.

FIG. 8 shows a tibial implant 8 in a view from below onto the surface region 12 that is in contact with the bone in the implanted state. According to FIG. 8, the tibial implant 8 comprises a continuous surface region 12. However, the surface region 12 can also be subdivided into a plurality of individual surface regions 12. The surface region 12 can comprise a surface structure which is designed analogously to the three-dimensional surface structures shown in FIG. 2 to 7 or further three-dimensional surface structures. In the example, the surface structure comprises a rib structure 26, having ribs 28, 28-1, 28-2 and rib portions 30 which extend obliquely with respect to a reference axis 34. Furthermore, a complete border is provided in an edge region 36 by a circumferential rib 28-3.

1. A femoral implant part for an orthopedic knee joint prosthesis, the femoral implant part comprising at least one surface region configured to face bone and be in contact with the bone in an implanted state, at least part of the femoral implant part being made from a plastics material, the femoral implant part comprising a three-dimensional surface structure in the at least one surface region, the three-dimensional surface structure comprising at least one rib structure, at least one first rib portion extending on a surface of the at least one surface region in a plane, or a first rib of the rib structure extending on the surface of the at least one surface region in the plane, and a second rib portion extending on the surface of the at least one surface region in the plane, or a second rib of the rib structure extending on the surface of the at least one surface region in the plane, extend in the plane in different directions, at least in portions.

2. The femoral implant part according to claim 1, wherein the three-dimensional surface structure comprises at least one intermediate space formed between at least two ribs and/or rib portions of the rib structure.

3. The femoral implant part according to claim 1, wherein, with respect to a reference axis extending in the plane, a rib or a rib portion extends in the plane longitudinally with respect to the reference axis, or transversely to the reference axis, or obliquely with respect to the reference axis, or in a curve or at an angle.

4. The femoral implant part according to claim 1, wherein at least two or more ribs or at least two or more rib portions of the rib structure form at least one intersection.

5. The femoral implant part according to claim 1, wherein ribs or rib portions of the rib structure are arranged according to a honeycomb pattern such that in each case six ribs or rib portions delimit a cell of the honeycomb pattern.

6. The femoral implant part according to claim 1, wherein a rib or a rib portion has a spiral profile in the plane.

7. A method for producing a femoral implant part according to claim 1, wherein the femoral implant part is produced by injection molding or additive manufacturing.

8. A tibial implant part for an orthopedic knee joint prosthesis, the tibial implant part comprising at least one surface region configured to face and be in contact with bone in an implanted state, at least part of the tibial implant part being made from a plastics material, the tibial implant part comprising a three-dimensional surface structure in the surface region, the three-dimensional surface structure comprising at least one rib structure, at least one first rib portion extending on a surface of the surface region in a plane or a first rib of the rib structure extending on the surface of the surface region in the plane, and a second rib portion extending on the surface of the surface region in the plane, or a second rib of the rib structure extending on the surface of the surface region in the plane, extend in the plane in different directions, at least in portions.

9. The tibial implant part according to claim 8, wherein the three-dimensional surface structure comprises at least one intermediate space formed between at least two ribs and/or rib portions of the rib structure.

10. The tibial implant part according to claim 8, wherein, with respect to a reference axis extending in the plane, a rib or a rib portion extends in the plane longitudinally with respect to the reference axis, or transversely to the reference axis, or obliquely with respect to the reference axis, or in a curve or at an angle.

11. The tibial implant part according to claim 8, wherein at least two or more ribs or at least two or more rib portions of the rib structure form at least one intersection.

12. The tibial implant part according to claim 8, wherein ribs or rib portions of the rib structure are arranged according to a honeycomb pattern such that in each case six ribs or rib portions delimit a cell of the honeycomb pattern.

13. The tibial implant part according to claim 8, wherein a rib or a rib portion has a spiral profile in the plane.

14. A method for producing a tibial implant part according to claim 8, wherein the tibial implant part is produced by injection molding or additive manufacturing.

15. An orthopedic knee joint prosthesis comprising:
a femoral implant part comprising at least one surface region configured to face bone and be in contact with the bone in an implanted state, at least part of the femoral implant part being made from a plastics material, the femoral implant part comprising a three-dimensional surface structure in the at least one surface region, the three-dimensional surface structure comprising at least one rib structure, at least one first rib portion extending on a surface of the at least one surface region in a plane, or a first rib of the rib structure extending on the surface of the at least one surface region in the plane, and a second rib portion extending on the surface of the at least one surface region in the plane, or a second rib of the rib structure extending on the surface of the at least one surface region in the plane, extend in the plane in different directions, at least in portions; and/or
a tibial implant part comprising at least one surface region configured to face and be in contact with bone in an implanted state, at least part of the tibial implant part being made from a plastics material, the tibial implant part comprising a three-dimensional surface structure in the surface region, the three-dimensional surface structure comprising at least one rib structure, at least one first rib portion extending on a surface of the surface region in a plane or a first rib of the rib structure extending on the surface of the surface region in the plane, and a second rib portion extending on the surface of the surface region in the plane, or a second rib of the rib structure extending on the surface of the surface region in the plane, extend in the plane in different directions, at least in portions.

16. The orthopedic knee joint prosthesis according to claim 15, wherein the orthopedic knee joint prosthesis comprises:
the femoral implant part;
the tibial implant part; and
a meniscus replacement part that forms a sliding partner for the femoral implant part and is held on the tibial implant part.
